(19) United States
(12) Patent Application Publication
(10) Pub. No.: US 2005/0143597 A1
(43) Pub. Date: Jun. 30, 2005

(54) Process for producing carbonyl compound

(76) Inventors: Eiichiro Mizushima, Ibaraki (JP); Teruyuki Hayashi, Chiba (JP); Kazuhiko Sato, Ibaraki (JP); Masato Tanaka, Tokyo (JP)

Correspondence Address: MORGAN LEWIS & BOCKIUS LLP, 1111 PENNSYLVANIA AVENUE NW, WASHINGTON, DC, 20004, US

(21) Appl. No.: 10/506,507

(22) PCT Filed: Mar. 6, 2003
(86) PCT No.: PCT/JP03/02650

(30) Foreign Application Priority Data
Mar. 6, 2002 (JP) 2002-060070
Aug. 30, 2002 (JP) 2002-256129

(51) Int. Cl. C07C 67/36; C07C 51/16; C07F 1/12

(52) U.S. Cl. 560/207; 562/544; 556/13

(57) ABSTRACT

The present invention relates to a process which comprises efficiently proceeding a hydration reaction of an alkyne in aspects of turnover numbers of a catalyst, yield and speed to thereby industrially and advantageously produce the corresponding carbonyl compound. The present invention provides a process for producing a carbonyl compound, which comprises reacting an alkyne compound with water in the presence of a gold catalyst which is an organogold complex compound and acid in an organic solvent.

### TECHNICAL FIELD

The present invention relates to a process for producing a carbonyl compound.

Organic carbonyl compounds have great industrial values, for example, various solvents, ketone resins of excellent light and chemical resistance, starting materials for radical polymerization initiators (ketone peroxides) to be used in producing synthetic resins, and the like. Also, since these compounds are widely used as starting materials or synthesis intermediates in producing various compounds such as drugs and pesticides, carbonyl compounds are extremely useful compounds.

### BACKGROUND ART

Heretofore, carbonyl compounds have been produced by a method such as condensation or oxidation of alcohol or a hydrocarbon. Although there is a known method which comprises hydrating an alkyne (an acetylene compound) in the presence of acid, no favorable result from the viewpoint of reactivity in practice can be obtained by this method unless using an alkyne which has been activated by an electron donating substituent such as ether, thioether or an amino group (J. March, Advanced Organic Chemistry, 4th ed., pp. 762-763).

There is known another method in which a mercury catalyst such as mercury nitrate or mercury acetate is used in an aqueous solution of an acid catalyst. This method is applicable to alkynes over a wider range in comparison with the case using an acid catalyst alone (P .F. Hudrlik and A. M. Hudrlik, The Chemistry of the Carbon-Carbon Triple Bond, Vol 1, S. Patai, ed., 1978, pp 240-243; G. W. Stacy and R. A. Mikulec, Organic Syntheses, 1963, Collect. Vol. 4, p. 13). In this method, however, it is needed to use mercury which should be avoided because of being an environmental pollutant. Furthermore, mercury should be used in a large amount of 5 to 10% by mmol based on a substrate and, moreover, the yield thus achieved is not sufficiently high. Thus, this method cannot be considered as an effective method for producing a carbonyl compound via a hydration reaction of an alkyne. In each of the method using an acid catalyst alone and the method using a combination of an acid catalyst with a mercury catalyst, only a low reactivity can be obtained and a large amount of acid is needed based on the starting alkyne. Therefore, it appears that these methods are not industrially advantageous.

As a method for avoiding use of mercury which is an environmental pollutant, methods using a catalyst containing a transition metal such as gold, rhodium, ruthenium, palladium or platinum are known. However, these methods cannot be considered as effective methods for producing a carbonyl compound via a hydration reaction of an alkyne, since catalytic efficiency and reaction yield still remain insufficient. In a hydration reaction of an alkyne using a catalyst containing trivalent gold disclosed by Y. Fukuda and K. Utimoto, J. Org. Chem., 56, 3729 (1991), for example, the catalytic efficiency is extremely low (turnover number of catalyst: about 50). In a method using a monovalent gold as a catalyst disclosed by J. H. Teles and M. Schulz (BASF AG), WO-A1 97/21648 (1997), the reaction yield is extremely low (lower than 10%). Attempts have been made to improve the catalyst efficiency and/or reaction yield in these methods using transition metal-containing catalysts by carrying out a reaction using a rhodium or ruthenium catalyst in the presence of acid serving as a cocatalyst. However, these methods cannot be regarded as industrially advantageous methods since the reactivity is low and a large amount of hydrochloric acid is needed (B. R. James and G. L. Rempel, J. Am. Chem. Soc., 91, 863 (1969); J. Harpern, B. R. James and A. L. W. Kemp, J. Am. Chem. Soc., 88, 5142 (1966)). Namely, it has been considered hitherto that no favorable effect can be obtained by a method in which a hydration reaction of an alkyne is carried out by adding a transition metal catalyst and acid. Accordingly, it has been required to obtain a process for producing a carbonyl compound via a hydration reaction of an alkyne at a high catalyst efficiency and a high reaction yield.

An object of the present invention is to provide a process which comprises efficiently proceeding a hydration reaction of an alkyne in aspects of turnover numbers of a catalyst, yield and speed to thereby industrially and advantageously produce the corresponding carbonyl compound.

### DISCLOSURE OF THE INVENTION

In order to achieve the above-described object, the present inventors have conducted intensive studies on a hydration reaction of an alkyne compound and, as a result, completed the present invention.

Accordingly, the present invention provides a process for producing a carbonyl compound as follows.
(1) A process for producing a carbonyl compound, which comprises reacting an alkyne compound with water in the presence of a gold catalyst which is an organogold complex compound and acid in an organic solvent.
(2) The process for producing a carbonyl compound according to the above item (1), wherein the alkyne compound is an alkyne compound represented by the following formula (1):
   R¹—C≡C—R² (1)
   wherein R¹ and R² each represents a hydrogen atom, an organic group, an organic oxy group, an organic oxycarbonyl group, an organic carbonyl group, an organic carbonyloxy group, an organic thio group, a silyl group, an organic group-substituted silyl group or a carboxyl group.
(3) The process for producing a carbonyl compound according to the above item (1), wherein the alkyne compound is an alkyne compound represented by the following formula (2):
   R¹—C≡C-A-C≡C—R² (2)
   wherein A represents a divalent organic group; and R¹ and R² each represents a hydrogen atom, an organic group, an organic oxy group, an organic oxycarbonyl group, an organic carbonyl group, an organic carbonyloxy group, an organic thio group, a silyl group, an organic group-substituted silyl group or a carboxyl group.
(4) The process according to any one of the above items (1) to (3), wherein the gold catalyst is a phosphine-gold complex compound represented by the following formula (3): 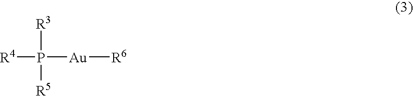
   wherein R³, R⁴ and R⁵ each represents an organic group or an organic oxy group; and R⁶ represents an organic group
(5) The process according to any one of the above items (1) to (4), wherein the organic solvent is alcohol.
(6) The process according to any one of the above items (1) to (5), wherein the reaction is carried out in the presence of a coordination additive.
(7) The process according to the above item (6), wherein the coordination additive is carbon monoxide.
(8) The process according to the above item (6), wherein the coordination additive is phosphite, phosphonite or phosphinite.

### BEST MODE FOR CARRYING OUT THE INVENTION

As the reaction material used in the present invention, alkyne compounds (acetylene compounds) over a wide range can be used. The alkyne compound used in the present invention includes an alkyne compound having a single alkynyl group as well as an alkyne compound having plural (from 2 to 4, preferably 2 or 3) alkynyl groups.

As the alkyne compound used in the present invention, alkyne compounds represented by the following formulae are advantageously used.
R¹—C≡C—R² (1)
R¹—C≡C-A-C≡C—R² (2)

In the above formulae, R¹ and R² each can represent an organic group. The organic group includes an aliphatic group having from 1 to 20 carbon atoms, an aromatic group having from 6 to 20 carbon atoms, and a heterocyclic group having from 5 to 20 elements constituting the ring.

The aliphatic group includes linear and cyclic groups, and saturated and unsaturated groups. The linear aliphatic group includes an alkyl group and an alkenyl group. The cyclic aliphatic group includes a cycloalkyl group and a cycloalkenyl group. In the alkyl group, the number of carbon atoms constituting the main chain is preferably from 1 to 10, and more preferably from 1 to 6.

In the alkenyl group, the number of carbon atoms constituting the main chain is preferably from 2 to 10, and more preferably from 2 to 6. The cycloalkyl group and the cycloalkenyl group can have either one or plural (from 2 to 4, preferably 2 or 3) rings. In such a group, the number of carbon atoms constituting the total carbon ring(s) in the molecule is from 3 to 20, and more preferably from 5 to 13.

The above-described aromatic group includes monocyclic and polycyclic groups. The polycyclic group includes a fused polycyclic group and a linear polycyclic group. More specifically, the aromatic group includes an aryl group and an aralkyl group.

The aryl group can have a monocyclic structure or a polycyclic structure, and the number of carbon atoms constituting the total carbon ring(s) in the molecule is from 6 to 20, and preferably from 6 to 16.

The aralkyl group can have a monocyclic structure or a polycyclic structure, and the number of carbon atoms constituting the total carbon ring(s) in the molecule is from 7 to 20, and preferably from 7 to 17.

The heterocyclic group includes an aliphatic heterocyclic group and an aromatic heterocyclic group. Ring-constituting elements constituting the heterocyclic group contain one or more hetero elements (oxygen, nitrogen, sulfur, selenium or the like).

The heterocyclic group can have a monocyclic structure or a polycyclic structure, and the number of elements constituting the total heterocyclic ring(s) in the molecule is from 5 to 20, and preferably from 5 to 13.

Examples of the aromatic heterocyclic group include groups derived from aromatic heterocycles such as a thiophene ring, a furan ring, a pyrole ring, a pyridine ring, a quinoxaline ring, a purine ring, an oxazole ring, a benzoxazole ring, a naphthoxazole ring, a thiazole ring, a benzothiazole ring, a naphthothiazole ring, a selenazole ring, a benzoselenazole ring, a naphthoselenazole ring, an imidazole ring, a benzimidazole ring, a naphthoimidazole ring, a quinoline ring, a quinoxaline ring, a purine ring, an acridine ring and a phenanthroline ring.

Examples of the aliphatic heterocyclic group include groups derived from aliphatic heterocycles such as a pyrazoline ring, a pyrralizine ring, a piperidine ring, an indoline ring, a morpholine ring, a pyran ring, an imidazolidine ring, a thiazoline ring, an imidazoline ring and an oxazoline ring.

Examples of the above-described organic group include methyl, ethyl, propyl, butyl, octyl, vinyl, propenyl, butynyl, hexenyl, octenyl, cyclohexyl, cyclohexylmethyl, cyclooctyl, cyclohexenyl, cyclooctyneyl, phenyl, tolyl, naphthyl, biphenyl, benzyl, phenethyl and naphthylmethyl, and heterocyclic groups derived from various heterocycles as described above.

In the formulae (1) and (2), R¹ and R² each can be an organic oxy group. The kind and examples of the organic group in the organic oxy group are the same as the groups described above. Preferred examples of the organic oxy group include an alkoxy group and an aryloxy group.

An alkyl group in the above-described alkoxy group includes linear and cyclic alkyl groups. In the case of the linear alkyl group, the number of carbon atoms constituting the main chain of the linear alkyl group is from 1 to 10, and preferably from 1 to 6. In the case of the cyclic alkyl group, the cyclic alkyl group can have a monocyclic or polycyclic structure, and the number of carbon atoms constituting the carbon ring is from 3 to 20, and preferably from 3 to 13.

An aryl group in the above-described aryloxy group can have a monocyclic or polycyclic structure, and the number of carbon atoms constituting the carbon ring(s) is from 6 to 19, and preferably from 6 to 16.

In the formulae (1) and (2), R¹ and R² each can be an organic oxycarbonyl group. The kind and examples of the organic group in the organic oxycarbonyl group are the same as the groups described above. Preferred examples of the organic oxycarbonyl group include an alkoxycarbonyl group and an aryloxycarbonyl group. In this case, an alkyl group in the alkoxycarbonyl group and an aryl group in the aryloxycabronyl group are the same as the groups described above with respect to the alkoxy group and the aryloxy group.

In the formulae (1) and (2), R¹ and R² each can be an organic carbonyl group. The kind and examples of the organic group in the organic carbonyl group are the same as the groups described above. Preferred examples of the organic carbonyl group include an alkylcarbonyl group and an arylcarbonyl group. In this case, an alkyl group in the alkylcarbonyl group and an aryl group in the arylcabronyl group are the same as the groups described above with respect to the alkoxy group and the aryloxy group.

In the formulae (1) and (2), R¹ and R² each can be an organic carbonyloxy group. The kind and examples of the organic group in the organic carbonyloxy group are the same as the groups described above. Preferred examples of the organic carbonyloxy group include an alkylcarbonyloxy group and an arylcarbonyloxy group. In this case, an alkyl group in the alkylcarbonyloxy group and an aryl group in the an arylcarbonyloxy group are the same as the groups described above with respect to the alkoxy group and the aryloxy group.

In the formulae (1) and (2), R¹ and R² each can be an organic thio group. The kind and examples of the organic group in the organic thio group are the same as the groups described above. Preferred examples of the organic thio group include an alkylthio group and an arylthio group. In this case, an alkyl group in the alkylthio group and an aryl group in the arylthio group are the same as the groups described above with respect to the alkoxy groups and the aryloxy groups.

In the formulae (1) and (2), R¹ and R² each can be a substituted silyl group in which at least one hydrogen atom in a silyl group is substituted with an organic group. The kind and examples of the organic group in the substituted silyl group are the same as the groups described above. Preferred examples of the substituted silyl group include an alkyl-substituted silyl group and an aryl-substituted silyl group. In this case, an alkyl group in the alkyl-substituted silyl group and an aryl group in the aryl-substituted silyl group are the same as the groups described above with respect to the alkoxy group and the aryloxy group.

In the formula (2), A represents a divalent organic group. Examples of the divalent organic group in this case include groups derived from various organic groups as described above with respect to the formula (1) by eliminating one hydrogen atom. Preferred examples of the divalent organic group include an alkylene group and an arylene group.

Each of the organic groups constituting R¹ and R² and the organic group constituting A as described above can have a substituent(s) having no undesirable effect on the reaction. The substituents include various hydrocarbon groups as described above as well as a halogen atom, a hydroxyl group, a cyano group, a carboxyl group, an alkoxy group, an acyl group, an acyloxy group, an amino group, a formyl group, a silyl group, a carbonyl group, an ester group, and the like.

Examples of the above-described R¹ and R² include a hydrogen atom, a methyl group, a propyl group, a butyl group, a hexyl group, a phenyl group, a thienyl group, a benzyl group, a propenyl group, a cyclohexenyl group, a methoxy group, a phenoxy group, a trimethylsilyl group, an acetyl group, a carboxyl group, a methyl ester group, and the like.

Examples of alkynes suitable for the production process according to the present invention include unsubstituted acetylene, butyne, hexyne, octyne, phenylacetylene, diphenylacetylene, ethynylthiophene, cyclohexenylacetylene, propargyl alcohol, methyl propargyl ether, trimethylsilylacetylene, 3-hexyn-2-one, propiolic acid, methyl propiolate, and the like, although the present invention is not limited thereto. Alkyne compounds having at least 2 acetylene bonds per molecule such as diethynylbenzene, 1,5-hexadiyne and 1,8-nonadiyne can also be preferably used.

The amount of water used in this reaction is not particularly limited. In general, water is used in at least one equivalent per acetylene bond. It is preferably used in 1 to 500 equivalents.

As the gold catalyst used in the hydration reaction of the alkyne compound in the present invention, an organogold complex compound is used. In the present invention, it is particularly advantageous to use a phosphine gold complex compound represented by the following formula (3).

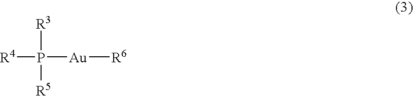

In the above formula, R³, R⁴ and R⁵ each represents an organic group or an organic oxy group. R⁶ represents an organic group. Examples of these organic groups include various organic groups and organic oxy groups described above with respect to the formulae (1) and (2).

The organic groups preferably used in the present invention include an alkyl group, a cycloalkyl group, an aryl group, an aralkyl group, and the like. Preferred examples of the organic oxy groups include an alkoxy group and an aryloxy group, and examples thereof include various groups described above.

Examples of the above-described R³, R⁴ and R⁵ include a methyl group, an ethyl group, a cyclohexyl group, a phenyl group, a benzyl group, a methoxy group, a phenoxy group, and the like. Examples of the above-described R⁶ include a methyl group, an ethyl group, a propyl group, a trifluoromethyl group, a cyclohexyl group, an ethynyl group, a phenyl group, a benzyl group, and the like.

Examples of the above-described phosphine gold compound include methyl(triphenylphosphine)gold, ethyl(triphenylphosphine)gold, propyl(triphenylphosphine)gold, trifluoromethyl(triphenylphosphine)gold, formylmethyl(triphenylphosphine)gold, acetylmethyl(triphenylphosphine)gold, pentafluorophenyl(triphenylphosphine)gold, phenylacetylide(triphenylphosphine)gold, methyl(trimethylphosphine)gold, methyl(triethylphosphine)gold, methyl(dimethylphenylphosphine)gold, methyl(diphenylmethylphosphine)gold, methyl(trimethylphosphite)gold, and the like, although the present invention is not limited thereto.

In the present invention, in addition to the above-described organic phosphine gold complex compounds, it is also possible to use organogold complex compounds such as chlorocarbonylgold(I), dimethyl(acetylacetonate)gold(III), chloro(triphenylphosphine)gold, chloro(cyclohexylisocyanide)gold, chloro(cyclooctene)gold, lithium dimethylaurate, lithium tetramethylaurate, trimethylgold, trimethyl(triphenylphosphine)gold, dichlorotetramethyldigold, dibromotetramethyldigold, and the like.

In the present invention, the gold catalyst is used in an amount sufficient to accelerate the hydration reaction of the alkyne compound, i.e., a so-called catalytic amount. In general, it is sufficient to use the gold catalyst at a ratio of 5% by mol or less, usually from 0.0001 to 2% by mol, in terms of metallic gold, per acetylene bond.

In the present invention, the gold catalyst is used in an amount sufficient to accelerate the hydration reaction of the alkyne compound, i.e., a so-called catalytic amount. In general, it is sufficient to use the gold catalyst at a ratio of 5% by mol or less, usually from 0.0001 to 2% by mol, in terms of metallic gold, per acetylene bond.

In the present invention, acid is used as a cocatalyst in addition to the gold catalyst. As the acid serving as a cocatalyst, various inorganic acids and organic acids which are known in public can be used. Examples of acid suitable for the production process according to the present invention include sulfuric acid, nitric acid, trifluoromethanesulfonic acid, methanesulfonic acid, perchloric acid, fluoroboric acid, fluorophosphoric acid, 12 tungsto(VI)-phosphoric acid hydrate, and the like, although the present invention is not limited thereto. Although such a catalyst may be used in large excess, it is usually used in a so-called catalytic amount. That is, the acid is used at a ratio of from 1 to 50% by mol based on the alkyne compound. In another advantageous embodiment, a high-molecular weight sulfonic acid such as Nafion is used.

The reaction of the present invention can be carried out in the atmosphere. Alternatively, it can be carried out in inert gas atmosphere such as nitrogen, argon or methane. In the present invention, the reaction can be further accelerated by adding carbon monoxide as a coordination additive. In the reaction of the present invention, carbon monoxide, which is used as an additive, can be used either as a substitute for inert gas or in the form of a gas mixture. The carbon monoxide pressure usually ranges from 0.01 to 100 kg/cm², although the present invention is not limited thereto.

In a still another preferred embodiment, the reaction of the present invention is carried out in the presence of a phosphite, phosphonite or phosphinite additive.

As the phosphite, a compound represented by the following formula (5) can be used.

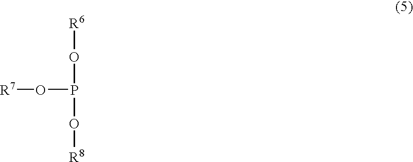

In the above formula, R⁷ to R⁹ each represents a group selected from organic groups. The kind and examples of the organic groups are the same as the groups described above. A preferable organic group is selected from an alkyl group, a cycloalkyl group, an aryl group and an aralkyl group. Examples thereof include the same groups as described above.

As the phosphonite, a compound represented by the following formula (6) can be used.

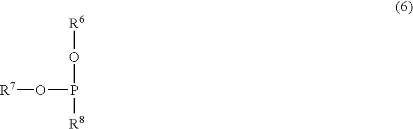

In the above formula, R⁷ to R⁹ have the same meanings as those defined in the above formula (5).

As the phosphinite, a compound represented by the following formula (7) can be used.

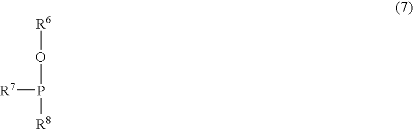

In the above formula, R⁷ to R⁹ have the same meanings as those defined in the above formula (5).

Examples of phosphite suitable for the production process according to the present invention include trimethylphosphite, triethylphosphite, triisopropylphosphite, trihexylphosphite, trioctylphosphite, tricyclohexylphosphite, triphenylphosphite, triorthotolylphosphite, methyldiphenylphosphite, trimethylolpropane phosphite, and the like, although the present invention is not limited thereto.

Examples of phosphonite suitable for the production process according to the present invention include dimethylphenylphosphonite, diisopropylphenylphosphonite, diphenylphenylphosphonite, diisopropylcyclohexylphosphonite, dimethylbutylphosphonite, and the like, although the present invention is not limited thereto.

Examples of phosphinite suitable for the production process according to the present invention include methyldiphenylphosphinite, ethyldiphenylphosphinite, phenyldiphenylphosphinite, p-methoxyphyenyldiphenylphosphinite, methyldiisopropylphosphinite, and the like, although the present invention is not limited thereto.

Although the amount used of the phosphite, phosphonite or phosphinite additive is not particularly limited, it is advantageous to use at least one equivalent of the additive to the gold catalyst.

The reaction of the present invention is carried out in an organic solvent. As the organic solvent, an alcohol solvent, an ether solvent, an ionic organic liquid or a polar organic solvent such as acetonitrile or dimethylformamide can be used.

Examples of the above-described alcohol solvent include alcohol having from 1 to 8, preferably from 1 to 6, carbon atoms, and preferred examples include methyl alcohol, propyl alcohol, butyl alcohol, and the like.

Examples of the above-described ether solvent include ethers having from 2 to 8, preferably from 3 to 6, carbon atoms, and preferred examples include dimethyl ether, diethyl ether, dioxane, and the like.

Examples of the above-described ionic organic liquid include organic borates such as 1-butyl3-methylimidazolium tetrafluoroborate, organic phosphates such as 1-butyl-3-methylimidazolium hexafluorophosphate, organic phosphates such as 4-methyl-N-butylpyridium hexafluorophosphate, quaternary ammonium salts such as methyltrioctylammonium chloride and methyltrioctylammonium hydrogen sulfate, and the like.

In view of the purpose of homogenizing the catalyst and the reaction materials and thus achieving a high catalytic activity, alcohol solvents such as methanol are particularly preferred as the organic solvent used in the present invention. The reaction cannot proceed at an advantageous speed at an extremely low temperature, while the catalyst would decompose at an extremely high temperature. Therefore, the reaction temperature is usually selected from the range from room temperature to 200° C., and the reaction is preferably carried out at a temperature from room temperature to 150° C. The solvent is used at a ratio of 30 to 10,000 parts by weight, preferably 50 to 1,000 parts by weight, per 100 parts by weight of the starting alkyne compound.

Water used as a reaction material in the present invention is used at a ratio of. 1 to 1,000 mol, preferably 1 to 500 mol, per mol of alkynyl bond contained in the alkyne compound. The acid is used at a ratio of 0.001 to 10 mol, preferably 0.01 to 0.5 mol, per mol of alkynyl group contained in the alkyne compound.

In the present invention, the organic solvent is used together with the organometal complex compound serving as a catalyst. In this case, the organic solvent exerts a cataytic effect of highly improving the activity and stability of the catalyst and thus elevating the yield of the target product.

According to the present invention, a carbonyl compound can be produced from an alkyne compound. In this carbonyl compound, carbon atoms contained in a triple bond in the starting alkyne compound have been carbonylated. The reaction formula thereof is as follows.
—C≡C—+H₂O →—C—CO— (or —CO—C—) (8)

### EXAMPLES

The present invention is described below in more detail by reference to the following examples, but the present invention is not limited to these Examples.

### Examples 1 to 4

To a solution in which 0.005 g of methyl(triphenylphosphine)gold (0.01 mmol) was dissolved in 1 ml of a solvent shown in Table 1, 0.11 g of 1-octyne (1 mmol) and an aqueous solution in which concentrated sulfuric acid (0.5 mmol) was dissolved in 0.5 ml of water were added. Table 1 summarizes the yields of 2-octanone obtained after stirring at 70° C. for 1 hour.

TABLE 1

| Example | Solvent | 2-Octanone yield (%) |
|---|---|---|
| 1 | methanol | 95 |
| 2 | 2-propanol | 71 |
| 3 | acetonitrile | 53 |
| 4 | dioxane | 56 |

### Example 5

A reaction was carried out in the same manner as in Example 1, except for using 1-butyl-3-methylimidazolium hexafluorophosphate as a solvent. As a result, 2-octanone was obtained in 89% yield.

### Example 6

A reaction was carried out in the same manner as in Example 5, except for using 0.01 g of methyl(triphenylphosphine)gold (0.02 mmol). As a result, 2-octanone was obtained in 96% yield.

### Example 7

A reaction was carried out in the same manner as in Example 1, except for using 0.01 g of methyl(triphenylphosphine)gold (0.02 mmol), using methyltrioctylammonium chloride as a solvent and carrying out the reaction for 12 hours. As a result, 2-octanone was obtained in 37% yield.

### Example 8

A reaction was carried out in the same manner as in Example 1, except for using 0.02 g of methyl(triphenylphosphine)gold (0.04 mmol), using methyltrioctylammonium hydrogen sulfate as a solvent and carrying out the reaction for 5 hours. As a result, 2-octanone was obtained in 75% yield.

### Comparative Example 1

A reaction was carried out in the same manner as in Example 1, except for using no concentrated sulfuric acid. In this case, the reaction did not proceed at all.

### Comparative Example 2

A reaction was carried out in the same manner as in Example 1, except for using no methyl(triphenylphosphine)gold. As a result, the reaction did not proceed at all.

### Comparative Example 3

A reaction was carried out in the same manner as in Example 1, except for using no organic solvent. In this case, the reaction did not substantially proceed.

### Example 9

A reaction was carried out in the same manner as in Example 1, except for using 0.001 g of methyl(triphenylphoshine)gold (0.002 mmol) and 0.22 g of 1-octyne (2 mmol). As a result, 2-octanone was obtained in 80% yield (catalyst turnover number: 800).

### Example 10

A reaction was carried out in the same as in Example 9, except for carrying out the reaction at a reaction temperature of 40° C. for 9 hours. As a result, 2-octanone was obtained in 75% yield (catalyst turnover number: 750).

### Example 11

To a solution in which 0.001 g of methyl(triphenylphosphine)gold (0.002 mmol) was dissolved in 10 ml of methanol, 2.2 g of 1-octyne (20 mmol) and an aqueous solution in which 0.05 g of concentrated sulfuric acid (0.5 mmol) was dissolved in 1 ml of water were added successively. After stirring at 70° C. for 1 hour, the yield of 2-octanone was 35% (catalyst turnover number: 3,500).

### Examples 12 to 14

A reaction was carried out in the same manner as in Example 11, except for using 0.5 mmol of trifluoromethanesulfonic acid (CF₃COOH), methanesulfonic acid (CH₃COOH) or 12 tungsto(VI)-phosphoric acid hydrate (H₃(PW₁₂O₄₀).nH₂O) instead of concentrated sulfuric acid. Table 2 summarizes the yields of 2-octanone and the catalyst turnover numbers.

TABLE 2

| | | 2-Octanone | Catalyst |
|---|---|---|---|
| Example | Kind of acid | yield (%) | turnover number |
| 12 | CF₃COOH | 99 | 9,900 |
| 13 | CH₃COOH | 77 | 7,700 |
| 14 | H₃(PW₁₂O₄₀).nH₂O | 80 | 8,000 |

### Example 15

To a solution in which 0.001 g of methyl(triphenylphosphine)gold (0.002 mmol) was dissolved in 3 ml of methanol, 0.62 g of Nafion-SAC13, 0.11 g of 1-octyne (1 mmol) and 0.5 ml of water were added successively. After stirring at 70° C. for 1 hour, the yield of 2-octanone was 92% (catalyst turnover number: 460).

### Example 16

To a solution in which 0.001 g of methyl(triphenylphosphine)gold (0.002 mmol) was dissolved in 10 ml of methanol, 4.4 g of 1-octyne (40 mmol) and an aqueous solution in which 0.1 g of trifluoromethanesulfonic acid (1 mmol) was dissolved in 2 ml of water were added successively. After stirring at 70° C. for 1 hour, the yield of 2-octanone was 70% (catalyst turnover number: 14,000).

### Example 17

A reaction was carried out in the same manner as in Example 11, except for carrying out the reaction in carbon monoxide gas atmosphere of 1 atm. As a result, the yield of 2-octanone was 99% (catalyst turnover number: 9,900).

### Example 18

To a solution in which 0.001 g of methyl(triphenylphosphine)gold (0.002 mmol) was dissolved in 10 ml of methanol, 4.4 g of 1-octyne (40 mmol) and an aqueous solution in which 0.15 g of trifluoromethanesulfonic acid (1 mmol) was dissolved in 2 ml of water were added successively, and the reaction was carried out in carbon monoxide gas atmosphere at 1 atm. After stirring at 70° C. for 1 hour, the yield of 2-octanone was 70% (catalyst turnover, number: 15,600).

### Example 19

A reaction was carried out in the same manner as in Example 11, except for using 0.001 g of methyl(triphenylphosphine)gold (0.002 mmol) and a solution in which 0.0013 g of trimethylphosphite (0.004 mmol) was dissolved in 10 ml of methanol, and carrying out the reaction at 70° C. for 5 hours. As a result, 2-octanone was obtained in 93% yield (catalyst turnover number: 9,300).

### Example 20

A reaction was carried out in the same manner as in Example 11, except for using 0.001 g of methyl(triphenylphosphine)gold (0.002 mmol) and a solution in which 0.0026 g of trimethylphosphite (0.02 mmol) was dissolved in 10 ml of methanol, and carrying out the reaction at 70° C. for 5 hours. As a result, 2-octanone was obtained in 94% yield (catalyst turnover number: 9,400).

### Example 21

A reaction was carried out in the same manner as in Example 11, except for using 0.001 g of methyl(triphenylphosphine)gold (0.002 mmol) and a solution in which 0.0025 g of ethyldiphenylphosphinite (0.01 mmol) was dissolved in 10 ml of methanol, and carrying out the reaction at 70° C. for 1 hour. As a result, 2-octanone was obtained in 64% yield (catalyst turnover number: 6,400).

The hydration reaction conditions as described above were applied to various starting materials other than 1-octyne to thereby carry out reactions. Next, Examples for individual substrates are described.

### Example 22

To a solution in which 0.001 g of methyl(triphenylphosphine)gold (0.002 mmol) was dissolved in 1 ml of methanol, 0.11 g of phenylacetylene (1 mmol) and an aqueous solution in which 0.05 g of concentrated sulfuric acid (0.5 mmol) was dissolved in 0.5 ml of water were added. After stirring at 70° C. for 1 hour, the yield of acetophenone was 75% (catalyst turnover number: 375).

### Example 23

A reaction was carried out in the same manner as in Example 22, except for using 0.5 mmol of trifluoromethanesulfonic acid instead of concentrated sulfuric acid. As a result, acetophenone was obtained in 98% yield (catalyst turnover number: 490).

### Example 24

To a solution in which 0.001 g of methyl(triphenylphosphine)gold (0.002 mmol) was dissolved in 10 ml of methanol, 2.1 g of phenylacetylene (20 mmol) and an aqueous solution in which 0.05 g of concentrated sulfuric acid (0.5 mmol) was dissolved in 1 ml of water were added. After stirring at 70° C. for 1 hour, the yield of acetophenone was 14% (catalyst turnover number: 1,400)

### Example 25

A reaction was carried out in the same manner as in Example 24, except for carrying out the reaction in carbon monoxide gas atmosphere at 1 atm. As a result, acetophenone was obtained in 33% yield (catalyst turnover number: 3,300).

### Example 26

To a solution in which 0.001 g of methyl(triphenylphosphine)gold (0.002 mmol) was dissolved in 1 ml of methanol, 0.12 g of 4-ethynyltoluene (1 mmol) and an aqueous solution in which 0.05 g of concentrated sulfuric acid (0.5 mmol) was dissolved in 0.5 ml of water were added. After stirring at 70° C. for 1 hour, the yield of p-methylacetophenone was 45% (catalyst turnover number: 225).

### Example 27

A reaction was carried out in the same manner as in Example 26, except for using 0.5 mmol of trifluoromethanesulfonic acid instead of concentrated sulfuric acid. As a result, p-methylacetophenone was obtained in 96% yield (catalyst turnover number: 480).

### Example 28

To a solution in which 0.001 g of methyl(triphenylphosphine)gold (0.002 mmol) was dissolved in 1 ml of methanol, 0.13 g of o-anisylacetylene (1 mmol) and an aqueous solution in which 0.05 g of concentrated sulfuric acid (0.5 mmol) was dissolved in 0.5 ml of water were added. After stirring at 70° C. for 1 hour, the yield of 2′-methoxyacetophenone was 95% (catalyst turnover number: 475).

### Example 29

To a solution in which 0.001 g of methyl(triphenylphosphine)gold (0.002 mmol) was dissolved in 1 ml of methanol, 0.13 g of m-anisylacetylene (1 mmol) and an aqueous solution in which 0.05 g of concentrated sulfuric acid (0.5 mmol) was dissolved in 0.5 ml of water were added. After stirring at 70° C. for 1 hour, the yield of m-methoxyacetophenone was 24% (catalyst turnover number: 120).

### Example 30

A reaction was carried out in the same manner as in Example 29, except for using 0.005 g of methyl(triphenylphosphine)gold (0.01 mmol). As a result, m-methoxyacetophenone was obtained in 77% yield (catalyst turnover number: 77).

### Example 31

To a solution in which 0.001 g of methyl(triphenylphosphine)gold (0.002 mmol) was dissolved in 1 ml of methanol, 0.13 g of p-anisylacetylene (1 mmol) and an aqueous solution in which 0.05 g of concentrated sulfuric acid (0.5 mmol) was dissolved in 0.5 ml of water were added. After stirring at 70° C. for 1 hour, the yield of p-methoxyacetophenone was 93% (catalyst turnover number: 465).

### Example 32

To a solution in which 0.005 g of methyl(triphenylphosphine)gold (0.01 mmol) was dissolved in 1 ml of methanol, 0.14 g of p-chlorophenylacetylene (1 mmol) and an aqueous solution in which 0.05 g of concentrated sulfuric acid (0.5 mmol) was dissolved in 0.5 ml of water were added. After stirring at 70° C. for 1 hour, the yield of p-chloroacetophenone was 54% (catalyst turnover number: 54).

### Example 33

To a solution in which 0.001 g of methyl(triphenylphosphine)gold (0.002 mmol) was dissolved in 1 ml of methanol, 0.14 g of o-chlorophenylacetylene (1 mmol) and an aqueous solution in which 0.05 g of concentrated sulfuric acid (0.5 mmol) was dissolved in 0.5 ml of water were added. After stirring at 70° C. for 1 hour, the yield of o-chloroacetophenone was 66% (catalyst turnover number: 330).

### Example 34

To a solution in which 0.001 g of methyl(triphenylphosphine)gold (0.002 mmol) was dissolved in 1 ml of methanol, 0.11 g of 5-hexynenitrile (1 mmol) and an aqueous solution in which 0.05 g of concentrated sulfuric acid (0.5 mmol) was dissolved in 0.5 ml of water were added. After stirring at 70° C. for 1 hour, the yield of 5-oxohexanitrile was 83% (catalyst turnover number: 465).

### Example 35

To a solution in which 0.001 g of methyl(triphenylphosphine)gold (0.002 mmol) was dissolved in 1 ml of methanol, 0.09 g of 1-hexyne (1 mmol) and an aqueous solution in which 0.05 g of concentrated sulfuric acid (0.5 mmol) was dissolved in 0.5 ml of water were added. After stirring at 60° C. for 2 hours, the yield of 2-hexanone was 99% (catalyst turnover number: 495).

### Example 36

To a solution in which 0.001 g of methyl(triphenylphosphine)gold (0.002 mmol) was dissolved in 3 ml of methanol, 0.09 g of 2-hexyne (1 mmol) and an aqueous solution in which 0.05 g of concentrated sulfuric acid (0.5 mmol) was dissolved in 0.5 ml of water were added. After stirring at 60° C. for 5 hours, the yield of 2-hexanone was 42%, and the yield of 3-hexanone was 34% (catalyst turnover number: 380).

### Example 37

To a solution in which 0.001 g of methyl(triphenylphosphine)gold (0.002 mmol) was dissolved in 3 ml of methanol, 0.42 g of 4-octyne (1 mmol) and an aqueous solution in which 0.05 g of concentrated sulfuric acid (0.5 mmol) was dissolved in 0.5 ml of water were added. After stirring at 70° C. for 5 hours, the yield of 4-octanone was 92% (catalyst turnover number: 460).

### Example 38

To a solution in which 0.005 g of methyl(triphenylphosphine)gold (0.01 mmol) was dissolved in 3 ml of methanol, 0.19 g of diphenylacetylene (1 mmol) and an aqueous solution in which 0.05 g of concentrated sulfuric acid (0.5 mmol) was dissolved in 0.5 ml of water were added. After stirring at 70° C. for 5 hours, the yield of 2-phenylacetophenone (deoxybenzoin) was 53% (catalyst turnover number: 53).

### Example 39

To a solution in which 0.005 g of methyl(triphenylphosphine)gold (0.01 mmol) was dissolved in 3 ml of methanol, 0.12 g of 1-phenyl-1-propyne (1 mmol) and an aqueous solution in which 0.05 g of concentrated sulfuric acid (0.5 mmol) was dissolved in 0.5 ml of water were added. After stirring at 70° C. for 5 hours, propiophenone was obtained in 45% yield, and benzyl methyl ketone was obtained in 30% yield (catalyst turnover number: 75).

### Example 40

A reaction was carried out in the same manner as in Example 39, except for using 0.001 g of methyl(triphenylphosphine)gold (0.002 mmol). As a result, propiophenone was obtained in 28% yield, and benzyl methyl ketone was obtained in 18% yield (catalyst turnover number: 230).

### Example 41

To a solution in which 0.005 g of methyl(triphenylphosphine)gold (0.01 mmol) and 0.0065 g of triphenylphosphite (0.02 mmol) were dissolved in 3 ml of methanol, 0.11 g of 5-chloro-1-pentyne (1 mmol) and an aqueous solution in which 0.05 g of concentrated sulfuric acid (0.5 mmol) was dissolved in 0.5 ml of water were added. After stirring the reaction mixture at 70° C. for 4 hours, the yield of 5-chloro-2-pentanone was 23% (catalyst turnover number: 23).

### Example 42

To a solution in which 0.005 g of methyl(triphenylphosphine)gold (0.01 mmol) was dissolved in 3 ml of methanol 0.11 g of 5-chloro-1-pentyne (1 mmol) and an aqueous solution in which 0.05 g of concentrated sulfuric acid (0.5 mmol) was dissolved in 0.5 ml of water were added, and the reaction was carried out in carbon monoxide gas atmosphere of 1 atm. After stirring at 70° C. for 4 hours, the yield of 5-chloro-2-pentanone was 72% (catalyst turnover number: 72).

### Example 43

To a solution in which 0.005 g of methyl(triphenylphosphine)gold (0.01 mmol) was dissolved in 1 ml of methanol, 0.10 g of 5-hexin-1-ol (1 mmol) and an aqueous solution in which 0.05 g of concentrated sulfuric acid (0.5 mmol) was dissolved in 0.5 ml of water were added, and the reaction was carried out in carbon monoxide gas atmosphere of 1 atm. After stirring at 70° C. for 3 hours, the yield of 6-hydroxy-2-hexanone was 33% (catalyst turnover number: 33).

### Example 44

To a solution in which 0.01 g of methyl(triphenylphosphine)gold (0.02 mmol) was dissolved in 2 ml of methanol, 0.18 g of 2-methyl-3-butin-2-ol (2 mmol) and an aqueous solution in which 0.05 g of concentrated sulfuric acid (0.5 mmol) was dissolved in 0.5 nil of water were added. After stirring at 70° C. for 2 hours, 3-hydroxy-3-methyl-2-butanone was obtained in 44% yield, and 3-methyl-2-butenal was obtained in 20% yield (catalyst turnover number: 64).

### Example 45

To a solution in which 0.01 g of methyl(triphenylphosphine)gold (0.02 mmol) was dissolved in 2 ml of methanol, 0.26 g of 1-ethynyl-1-cyclohexanol (2 mmol) and an aqueous solution in which 0.30 g of 12 tungsto(VI)-phosphoric acid hydrate (H₃_{(PW}₁₂O₄₀)nH₂O) (0.1 mmol) was dissolved in 0.5 ml of water were added. After stirring at 70° C. for 2 hours, 1-acetyl-1-cyclohexanol was obtained in 45% yield, and cyclohexylidene acetaldehyde was obtained in 17% yield (catalyst turnover number: 62).

### Example 46

To a solution in which 0.0024 g of methyl(triphenylphosphine)gold (0.005 mmol) was dissolved in 3 ml of methanol, 0.13 g of 1,4-diethynylbenzene (1 mmol) and an aqueous solution in which 0.05 g of concentrated sulfuric acid (0.5 mmol) was dissolved in 0.5 ml of water were added. After stirring at 70° C. for 2 hours, 4-ethynylacetophenone was obtained in 65% yield, and 1,4diacetylbenzene was obtained in 18% yield (catalyst turnover number: 202).

### Example 47

To a solution in which 0.01 g of methyl(triphenylphosphine)gold (0.002 mmol) was dissolved in 3 ml of methanol, 0.13 g of 1,8-nonadiyn (1 mmol) and an aqueous solution in which 0.05 g of concentrated sulfuric acid (0.5 mmol) was dissolved in 0.5 ml of water were added. After stirring at 70° C. for 2 hours, 2,8-nonadione was obtained in 99% yield (catalyst turnover number: 990).

### Example 48

To a solution in which 0.01 g of methyl(triphenylphosphine)gold (0.002 mmol) was dissolved in 0.6 ml of methanol, 0.023 g of 2-ethynylthiophene (0.2 mmol) and an aqueous solution in which 0.01 g of concentrated sulfuric acid (0.1 mmol) was dissolved in 0.1 ml of water were added. After stirring at 70° C. for 1 hour, the yield of 2-acetylthiophene was 92% (catalyst turnover number: 92).

### Industrial Applicability

According to the present invention, carbonyl compounds, which have great industrial values and are highly useful as fine chemicals for drugs, pesticides, etc., can be efficiently produced. In the present invention, the reaction proceeds very efficiently in comparison with the existing methods, which makes the process according to the present invention highly excellent from an economical viewpoint. Thus, the present invention has great industrial meanings.

1. A process for producing a carbonyl compound, which comprises reacting an alkyne compound with water in the presence of a gold catalyst which is an organogold complex compound and acid in an organic solvent.

2. The process for producing a carbonyl compound according to claim 1, wherein the alkyne compound is an alkyne compound represented by the following formula (1):
R¹—C≡C—R² (1)
wherein R¹ and R² each represents a hydrogen atom, an organic group, an organic oxy group, an organic oxycarbonyl group, an organic carbonyl group, an organic carbonyloxy group, an organic thio group, a silyl group, an organic group-substituted silyl group or a carboxyl group.

3. The process for producing a carbonyl compound according to claim 1, wherein the alkyne compound is an alkyne compound represented by the following formula (2):
R¹—C≡C-A-C≡C—R (2)
wherein A represents a divalent organic group; and R¹ and R² each represents a hydrogen atom, an organic group, an organic oxy group, an organic oxycarbonyl group, an organic carbonyl group, an organic carbonyloxy group, an organic thio group, a silyl group, an organic group-substituted silyl group or a carboxyl group.

4. The process according to any one of claims 1 to 3, wherein the gold catalyst is a phosphine-gold complex compound represented by the following formula (3): 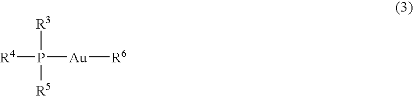
wherein R³, R⁴ and R⁵ each represents an organic group or an organic oxy group; and R⁶ represents an organic group.

5. The process according to any one of claims 1 to 4, wherein the organic solvent is alcohol.

6. The process according to any one of claims 1 to 5, wherein the reaction is carried out in the presence of a coordination additive.

7. The process according to claim 6, wherein the coordination additive is carbon monoxide.

8. The process according to claim 6, wherein the coordination additive is phosphite, phosphonite or phosphinite.
